(19) United States
(12) Patent Application Publication
(10) Pub. No.: US 2024/0206974 A1
(43) Pub. Date: Jun. 27, 2024

(54) TRANSTHORACIC ROUTE PLANNING SYSTEM AND METHOD

(71) Applicant: Broncus Medical Inc., San Jose, CA (US)

(72) Inventors: Matthew McTaggart, Grand Cayman (KY); Kun-Chang Yu, San Jose, CA (US)

(21) Appl. No.: 18/536,333

(22) Filed: Dec. 12, 2023

Related U.S. Application Data
(60) Provisional application No. 63/435,009, filed on Dec. 23, 2022.

(51) Int. Cl. A61B 34/10; A61B 34/20; G06T 7/00; G06T 7/10

(52) U.S. Cl. CPC A61B 34/10; A61B 34/20; G06T 7/0012; G06T 7/10; A61B 2034/105; A61B 2034/107; A61B 2034/2065; G06T 2200/04; G06T 2200/08; G06T 2200/24; G06T 2207/10081

(57) ABSTRACT

A method for automatically planning a transthoracic route from a candidate location on the skin of a patient to a target nodule in the patient on which to perform a procedure. The method comprises identifying a plurality of candidate skin locations on the skin of the patient and automatically computing a route to the target nodule from each candidate skin location. A score is automatically computed for each candidate skin location based on (a) route characteristics, (b) procedural parameters, and (c) obstacle clearance along the route. Related systems are also described.

### CROSS-REFERENCE TO RELATED APPLICATIONS

This claims priority to provisional application No. 63/435,009, filed Dec. 23, 2022, entitled “TRANSTHORACIC ROUTE PLANNING SYSTEM AND METHOD” the entirety of which is incorporated by reference in its entirety for all purposes.

### FIELD OF THE INVENTION

The present invention relates to surgical procedures directed to the thorax and more particularly, to transthoracic route planning.

### BACKGROUND OF THE INVENTION

Transthoracic surgical procedures such as transthoracic needle aspiration (TTNA) are surgical procedures in which the approach is through the thoracic cavity or chest wall. Under sterile conditions, local anesthesia, and imaging guidance—usually CT but sometimes ultrasonography for pleural-based lesions—a biopsy needle is passed into the suspected lesion while patients hold their breath. Two or 3 samples may be collected for cytologic and bacteriologic processing. After the procedure, fluoroscopy and chest x-rays are used to rule out pneumothorax and hemorrhage.

There are several challenges associated with performing a TTNA including avoiding bones (e.g., the ribs, spine, and scapula), vessels (e.g., the aorta, and PA), heart, airways, lymph nodes, as well as not puncturing the fissures between the lobes.

Another challenge of TTNA is the risk of pneumothorax. Pneumothorax, also known as collapsed lung, arises when air enters the pleural cavity. The air pushes on the outside of the lung, making it collapse. It can be a serious complication, leading to hemodynamic compromise unless medical intervention is provided.

Current solutions include physician technique to plan and perform the procedure. Such a heavy reliance on the physician skill, however, is undesirable because of the variability in skill and experience between physicians.

In view of the above, a planning system and method that addresses the above challenges is desired.

### SUMMARY OF THE INVENTION

Systems and methods are described for computing candidate skin locations, and in embodiments, a 3D location and direction (namely, pose) for performing transthoracic ablation and biopsy.

In embodiments, imaging information and data are input or received by the system. Candidate skin locations for ablation and biopsy are identified. A route is computed for each candidate skin location to the target nodule. Each candidate skin location is scored based on route characteristics, procedural parameters, and obstacle clearance.

Optionally, highest-ranking candidate skin locations for biopsy and ablation are displayed to the physician.

In embodiments, the physician marks the target nodule to be used in the route computation.

In embodiments, biopsy routes having the shortest needle depth are weighted higher than other candidate routes.

In embodiments, ablation treatment routes that position the major axis of the electrode parallel to the pleural surface closest to the target are weighted higher than other candidate routes.

The description, objects and advantages of the present invention will become apparent from the detailed description to follow, together with the accompanying drawings.

### BRIEF DESCRIPTION OF THE DRAWINGS

FIG. 1 is a block diagram of a system in accordance with an embodiment of the invention;

FIG. 2 is a flow chart illustrating a method for computing a transthoracic path for performing a procedure in accordance with an embodiment of the invention;

FIG. 3 shows a screen shot of a display during the step of annotating a target in accordance with an embodiment of the invention;

FIG. 4 shows a screen shot of a display during the step of manually identifying a skin location in accordance with an embodiment of the invention;

FIG. 5 is an illustration of the route, anatomy, target, and treatment electrode in accordance with an embodiment of the invention;

FIG. 6A is a 3D view of the anatomy and candidate skin locations scored for biopsy;

FIG. 6B is an enlarged view of portion of the view shown in FIG. 6A;

FIG. 6C is an enlarged view of another portion of the view shown in FIG. 6A;

FIG. 7A is a 3D view of the anatomy and candidate skin locations scored for ablation;

FIG. 7B is an enlarged view of a portion of the view shown in FIG. 7A;

FIG. 8 is a screen shot of a display showing different types of candidate paths for the user to select in accordance with an embodiment of the invention;

FIGS. 9-10 are fused-CT views displaying the biopsy path in accordance with an embodiment of the invention; and

FIGS. 11-12 are fused CT views displaying the treatment path in accordance with an embodiment of the invention.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. It is to be appreciated that unless defined otherwise, all technical and scientific terms used herein have the same meaning as commonly understood by one of ordinary skill in the art to which this invention belongs.

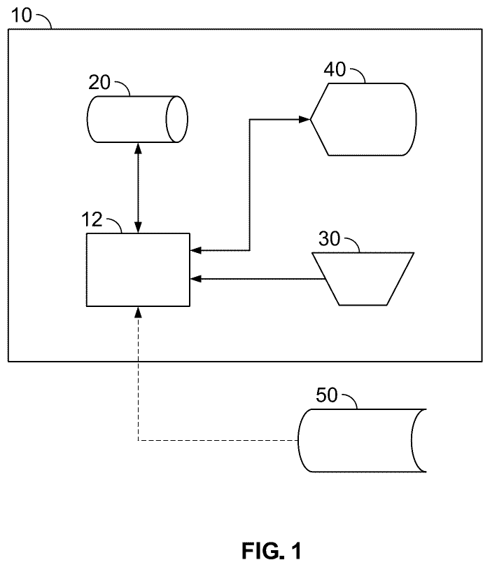

FIG. 1 illustrates a system 10 for automatically computing a percutaneous transthoracic path through the skin to a target.

The system 10 shown in FIG. 1 includes a processor 12 programmed and operable to compute candidate skin locations, and to compute optimal routes from the candidate skin locations to the target based on various data and information as described herein.

System 10 is shown having a storage device 20 which holds or stores information including various software programs or modules to compute the optimal routes to the target, as well as imaging, device, location, and procedural data.

The system 10 shown in FIG. 1 includes a user input device 30 such as, for example, a keyboard, joystick, or mouse. The user input device allows a user such as the physician to add or input data and information as well as to make modifications. For example, in embodiments, the system is operable to allow the physician to modify the skin or target location and to make notes (or annotate) in the files and records.

The system 10 shown in FIG. 1 also includes a display 40 which is operable with the processor to present reports, data, images, results and models in various formats including without limitation graphical, tabular, and pictorial form. In embodiments, the screen is a touchscreen type display and can be used to input information to the system.

The system may also comprise one or more ports, operable to communicate with an external storage device such as a USB stick, a CD, or other media storage device.

In another embodiment, the processor is connectable to a remote storage device 50 through the internet or through another communication line to access a network. For example, patient data CT scans may be stored on a server of a hospital and the processor of the instant application is adapted to access such data via a communication line and process the data.

Displays may be incorporated with the processor in an integrated system. In another embodiment, the displays cooperate with the processor from a remote location. A processor may be adapted to send or deliver data across a network to one or more displays, tablets, or portable computer devices or smart phones such as the iPhone® manufactured by Apple, Inc. Cupertino, CA, United States.

A wide range of chips and cards may be incorporated into the system including without limitation graphics cards, sound cards, wireless communication cards, etc. Indeed, although the computer system shown in FIG. 1 is shown having only a few components, the invention is not intended to be so limited. The invention is intended to be limited only as defined in the appended claims.

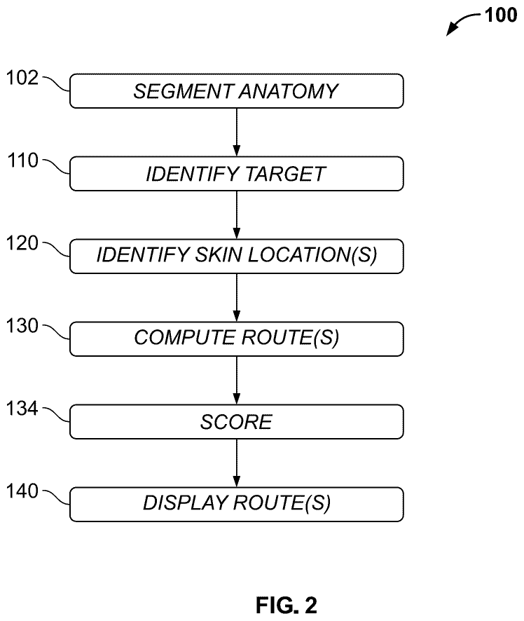

FIG. 2 shows a method or process 100 to compute a route from a candidate skin location to a target in accordance with an embodiment of the invention.

Step 102 recites to segment the anatomy. In embodiments, a segmentation module or program is stored on the system and is operable to segment the bone (e.g., ribs, spine, scapula), airways, blood vessels (e.g., PA, Aorta, PV, and peripheral vessels), heart, lobes (and fissures between or connecting the lobes), pleura, target (e.g., lung nodule, lymph node, or tumor), lymph nodes, and skin in the thoracic region based on available image data from a patient such as, e.g., high-resolution computed tomography (HRCT) or MRI scans. An exemplary technique to determine a 3D model of a bronchial tree anatomy is disclosed in U.S. Patent Publication No. US 2006/0159328 entitled “Method and apparatus for airway detection and segmentation”. An exemplary technique to determine a 3D model of the ribs and spine is disclosed in U.S. Pat. No. 10,872,415, entitled “Learning-based spine vertebra localization and segmentation in 3D CT”. An exemplary technique to determine a 3D model of vasculature in the thoracic anatomy is disclosed in U.S. Pat. No. 6,728,566 entitled “Vessel tracking and tree extraction method and apparatus”. Also, by use of the term “thoracic anatomy” it is meant to include, without limitation, the lungs, lymph nodes, scapula, ribs and spine, chest cavity, heart and vasculature (including all the vessels therein), lobes, fissures, pleura, and other tissues and structures in and around the vicinity of the thoracic region.

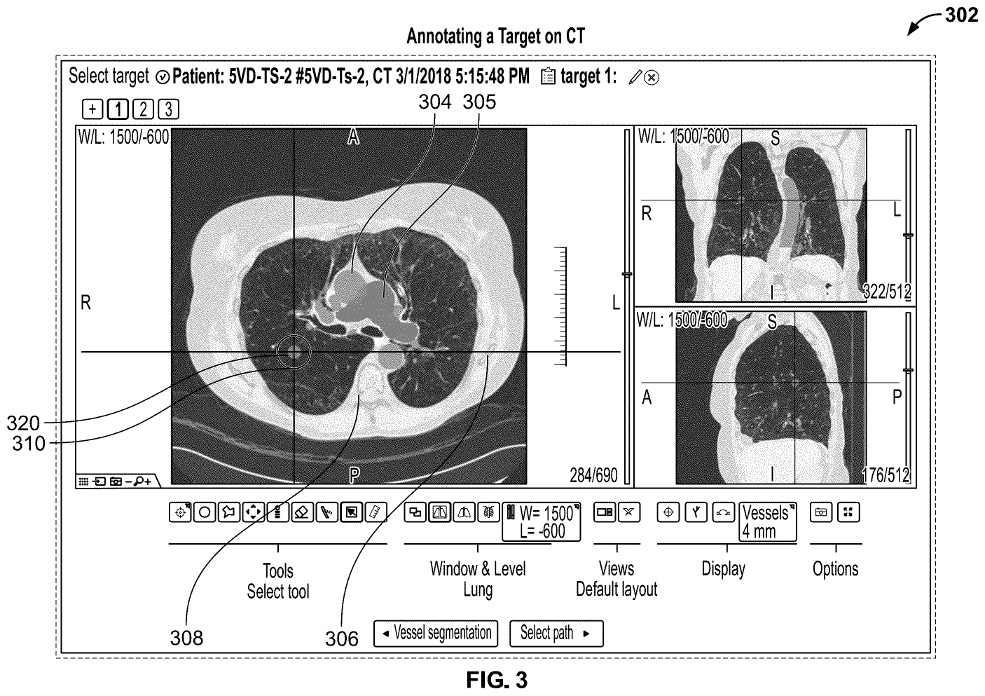

Step 110 recites to identify a target. In embodiments, the user annotates or marks the target. For example, and with reference to FIG. 3, a 2D transverse view 302 is shown fused with the segmented vessels (304,305) and bones (306,308). Such views can be generated or rendered using the process or rendering engine described herein and based on the segmented anatomy described above. The literature describes various techniques for rendering segmented objects, and fusing the objects with other images such as CT views and/or a digitally reproduced radiograph (DRR). See, for example, PCT Pub. No. WO2009/103046 to Higgins, filed Feb. 16, 2009, and entitled “Method and Apparatus for Organ Path Report Generation and Preview”; U.S. Pat. No. 10,872,415 to Cheng et al., filed Mar. 27, 2018, and entitled “LEARNING-BASED SPINE VERTEBRA LOCALIZATION AND SEGMENTATION IN 3D CT”; and U.S. Pat. No. 8,712,137 to Wollenweber, filed May 29, 2012, and entitled “Methods and system for displaying segmented images”, each of which is incorporated herein by reference in its entirety for all purposes.

A cross-hair tool 310 is controllable by an input device such as a mouse. The user can manipulate the mouse to hover the cross-hair tool 310 over the target 320 to select target. In embodiments, the user can mark the target in one or more of the different views. The selected target is stored.

With reference again to FIG. 2, step 120 recites to identify the skin location(s). The skin location is the location on the skin to create an opening, and insert the tools to perform the medical procedure. Examples of tools include, without limitation, a trocar, catheter, aspiration needle, RF ablation instrument, etc.

### Manually Selecting Skin Location(s)

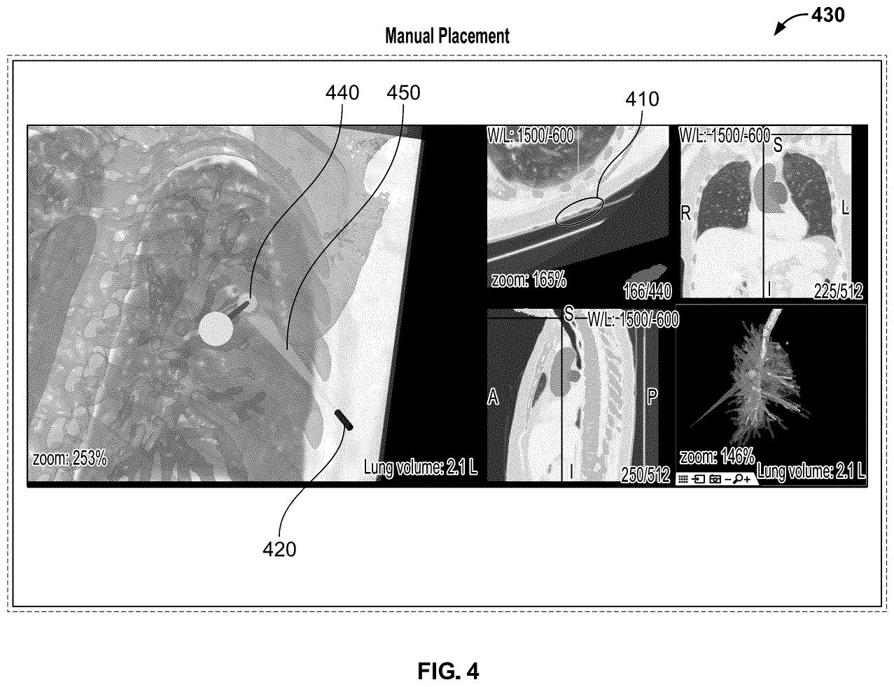

In one embodiment, the candidate skin locations are identified manually by the user. With reference to FIG. 4, the method provides a pointer tool 420 superimposed on the virtual view 430. Using an input device such as a mouse, the user can mark or place where on the skin the opening 410 is to be created. Several different views are also provided showing the skin location, and a computed path/route to the target mass 440 resulting in a cone-shaped route 450 from the skin location to the circumference of the target. However, it is to be understood the candidate route can be rendered and displayed in other shapes, formats and colors. The cone-shaped route 450 is only one example to illustrate an embodiment of the invention.

The processor is programmed and operable to compute the route in real time with minimal constraints (e.g., shortest path, no obstacles) between the demarcated skin location and the target. As the user moves/adjusts the skin location, the method provides an updated route to the target, and displays it in one or more of the virtual views. In embodiments, the system can compute a route passing through obstacles but provides warnings in the form of, e.g., real-time pop-ups or visual effects. An example of a visual effect in accordance with an embodiment of the invention is to display an indicator (e.g., a traffic light symbol) where red indicates to ‘avoid the obstacle’ (e.g., fissure, vessel); yellow indicates ‘exercise caution’ (e.g., bone); and green indicates ‘all clear’ (e.g., planned route appears free of obstacles).

The user can then save one or more of the candidate skin locations.

### Automatically Selecting Skin Location(s)

In a preferred embodiment, the skin locations are computed automatically. For example, a skin location computation engine can generate a plurality of candidate skin locations in areas in the vicinity of the target 440. For example, in embodiments, candidate skin locations are automatically computed based on whether the location is on the skin surface; which side of the patient the target is located; and whether the target be reached by a given instrument/needle from the skin location. Other skin locations may be excluded.

A plurality or array of candidate skin locations may be autonomically generated within the band-shaped region. In embodiments, the candidate skin locations are equidistance apart, preferably 0.5 to 2 mm, and in some embodiments 2 mm.

In embodiments, one or more fine tuning steps compute additional candidate skin locations near the best points from the initial computation. For example, where the initial candidate skin locations are generated according to a spacing of (S), the fine-tuning step generates a set of candidate skin locations near the best locations and according to an equidistant spacing of (S/2), where S may equal to the diameter of the biopsy needle (or another tool such as the ablation catheter). The fine tuning spacing is then used to compute the candidate skin locations and to find the best score.

In embodiments, the fine tuning spacing is similar to the X, Y, or Z dimension of a voxel corresponding to the 3D image. In embodiments, the fine tuning spacing is less than or equal to 0.5 mm.

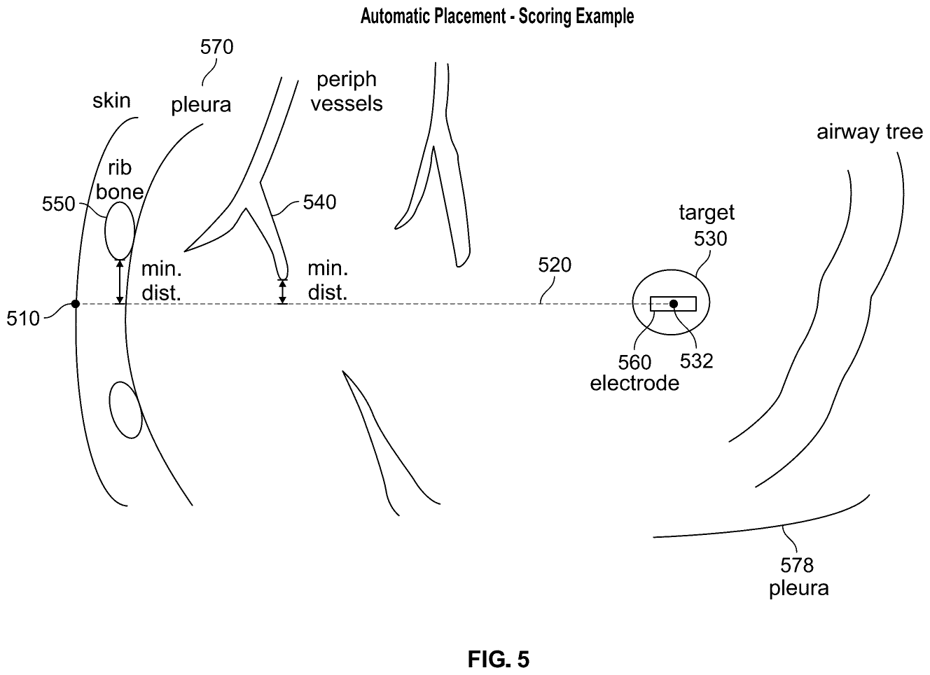

Step 130 states to compute the route(s). With reference to FIG. 5, in embodiments, for each candidate skin location 510, a route 520 to the target 530 is computed and scored based on different categories of information comprising (a) route characteristics, (b) obstacle clearance along the route, and (c) procedural parameters.

Examples of route characteristics include the length of the route. In embodiments, the length is computed as the shortest distance from the skin location 510 to the target 530 and more preferably, from the skin location 510 to the center of mass 532 of the target 530.

In other embodiments, the route can be straight or curved, have turns, or be comprised of multiple straight line segments.

Route characteristics can also include the angles of the needle relative to the CT. Examples of angles include roll, pitch and yaw. Additionally, although we refer to use of CT throughout the specification, it is to be understood that other means of generating 3D image data of the patient can be used with the present invention including, e.g., MRI, and 3D ultrasound.

Examples of obstacle clearance along the route include a minimum distance between the vessels 540 and the route to ensure the vessels are not impacted (e.g., injured, punctured, cut, or displaced) during the procedure. This constraint may be pre-stored in the system, or set and/or adjusted by the physician via, e.g., a keyboard. Minimum distances may vary and be based on the obstacle to be avoided. For example, critical delicate obstacles such as the aorta may be assigned a greater clearance than hard obstacles such as the ribs 550. In preferred embodiments, the system allows the physician to configure or adjust the minimum distances based on their preference for a safety margin. An exemplary range for the minimum distance is from 0.5 to 30 mm.

Procedural parameters include the characteristics and information related to the type of procedure and types of devices used in the procedure. Examples of device information are model and model number, and its specifications. Device information can include, for example, the total length, needle or ablation portion length, width or diameter, shape and curvature, and other features that may be useful planning a route as described herein.

In embodiments, needle depth for obtaining a tissue biopsy is computed. Needle depth is computed assuming the needle penetrates a substantial portion of the target, preferably until the tip reaches the back of the target.

Additionally, as discussed further herein, prior device data and/or profiles of the devices can be stored in a library in the computer storage. Each type of device can have a unique known profile including, for example, model identifier, the total length, diameter, shape, and other features that may be useful in planning a route. Design or CAD drawing files may be associated with the device profile. The profiles may also include specific/desirable views, skin location placement, angles of approach, and procedures for each type of device or, in the case such a profile or file does not yet exist, an instruction or command to prompt the user for creation of a new device file with specific procedures for the particular device. For example, the library may comprise a device file for each of a trocar or introducer sheath, RF ablation catheter and biopsy needle.

### Breathing Motion

In embodiments, optionally, we input a breathing motion profile corresponding to the respiratory cycle of the patient. This may be input or received in the form of, for example, image data of the bronchial tree and airways at multiple points in time corresponding to inhalation, exhalation, and perhaps one or more time points between inspiration and expiration. The data may be processed to identify displacement of tissues and tissue surfaces. A review of the image data across multiple points in time serves to accurately show the breathing motion profile of the patient. An exemplary process for carrying this out is described in “Fast Deformable Registration on the GPU: A CUDA Implementation of Demons” by Pinar Muyan-Ozcelik, 2008 wherein the authors describe using a pair of CT scans acquired from the same patient, one at a full level of inspiration and the second at a full level of expiration. The deformable registration technique described in this paper gives the mapping of each discrete point within the lungs at from its geometric location at expiration to inspiration and from inspiration to expiration. From these mappings, the location of any region within the chest can be estimated at points during the breathing cycle.

In other embodiments, lung motion is estimated based on placing external sensors on the chest of the patient and observing the motion of the sensors during breathing cycles. Examples of sensors include, without limitation, optical sensors or electromagnetic tracking sensors. Regardless of the technique for tracking breathing motion of the patient, the breathing motion can be input to the computing step for scoring, discussed herein.

### Scoring

With reference again to FIG. 2, step 134 recites score. In embodiments, given the previously-described information (e.g., route characteristics, type of procedure, distance from the route to an obstacle, device characteristics and orientation), the system can assign costs to the various types of information and then examine potential combinations of skin locations and routes.

A total weighted combinatorial cost/benefit for a particular skin location route may be determined or calculated. An example of a combinatorial cost/benefit algorithm is the genetic algorithm, which finds subsets of solution combinations (in this specific case, skin locations) from a large corpus of potential solutions as described by Goldberg in Genetic Algorithms in Search, Optimization, and Machine Learning, 1989. The system may then rank and return the skin location (or skin locations) with the best cost/benefit characteristics.

In embodiments of the invention, the individual components within the cost/benefit analysis can be weighted differently dependent upon the requirements of the procedure and the physician preferences.

### Biopsy Weighting

For example, when computing a biopsy skin location, the needle depth is weighted relatively higher than other components. Particularly, in a biopsy procedure, skin locations providing the shortest needle depth are desired.

### RF Ablation Weighting

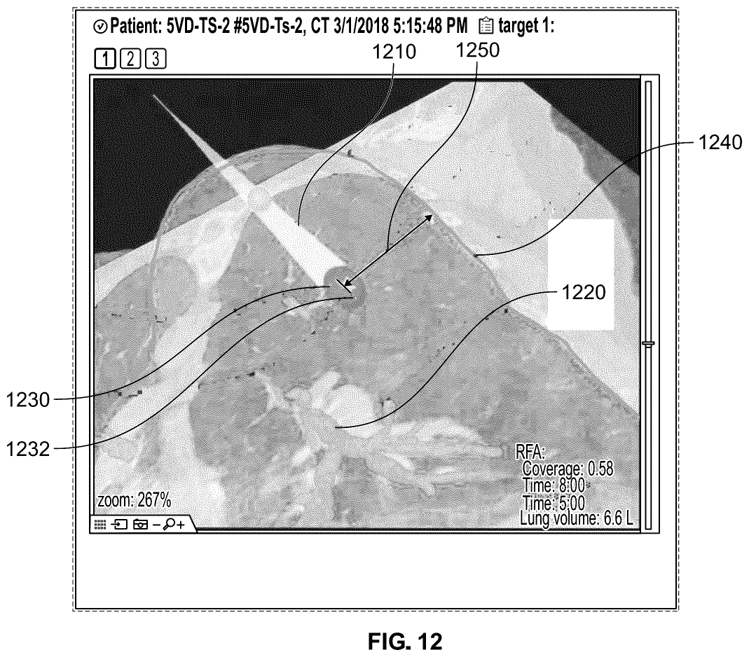

With reference again to FIG. 5, when computing a skin location 510 for treatment, the orientation of the electrode 560 relative to the closest pleura surface point 578 is weighted relatively higher than the other components. Particularly, and without intending to be bound to theory, we assume the ablation zone is longest along the electrode length and desire to maintain the ablation clear from the pleura. We weight routes placing the electrode major axis parallel to the closest pleura surface highest as shown in, e.g., FIG. 5 with reference to electrode 560 and closest pleura 578, and FIG. 12 with reference to electrode major axis 1232 and pleura 1240.

In embodiments, we also weight the distance from the back of the target to the closest pleura surface higher than other components to ensure the ablation zone does not damage the pleura during ablation.

Additionally, in embodiments, we apply an ablation model and compute tumor coverage and collateral damage for each route. In embodiments, we assume the ablation zone grows with time according to a power level. In embodiments, we model the shape of the ablation zone as an ellipsoid where x- and y-axis are equal, and the z-axis along electrode length is longest. However, in other embodiments, a trained machine learning model can be employed to compute tumor coverage and collateral damage. An ablation model is described in US Patent Publication No. 20210401502, entitled “IMAGE-GUIDED LUNG TUMOR PLANNING AND ABLATION SYSTEM.”

An exemplary output for the ablation model ranges from 0 to 1, where 0 means no coverage and 1 is full coverage.

Preferably, the model is adapted to weight towards complete tumor coverage with minimal collateral damage.

Based on the foregoing, an aggregate score can be computed for each skin location.

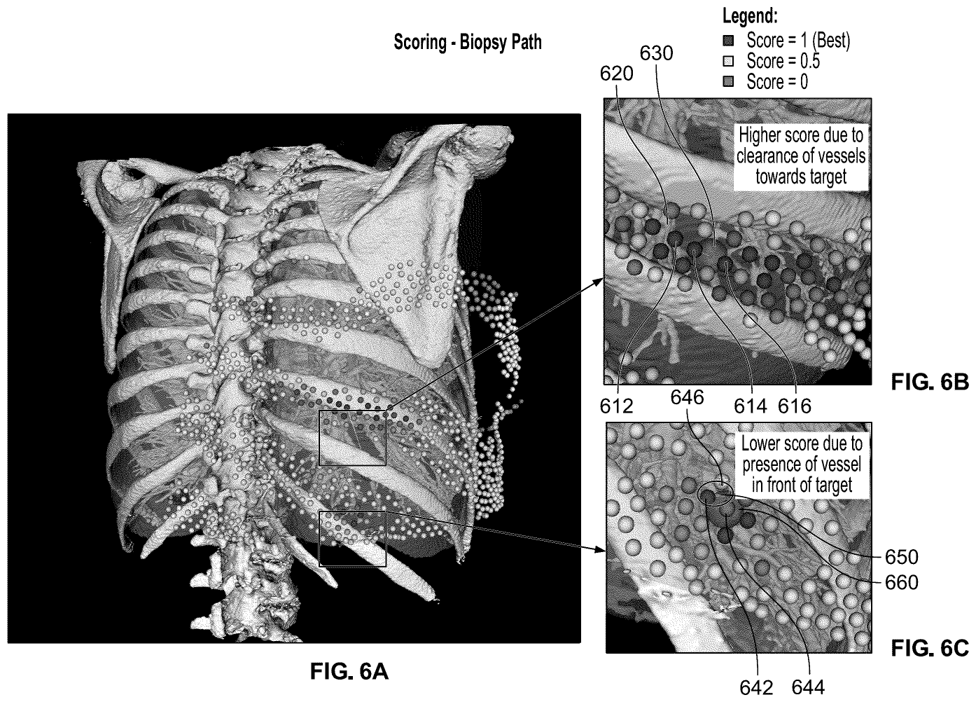

With reference to FIG. 6A, a biopsy path score is visually shown for each skin location where best scores are shown according to the legend. The enlarged view shown in FIG. 6B illustrates several skin locations 612, 614, 616 with high scores due to clearance of vessels 620 towards target 630.

In contrast, the enlarged view shown in the FIG. 6C shows lower scored skin location 646 due the presence of a vessel 650 in front of the target 660, whereas 642, 644 are slightly better for a better straight shot to the target 660.

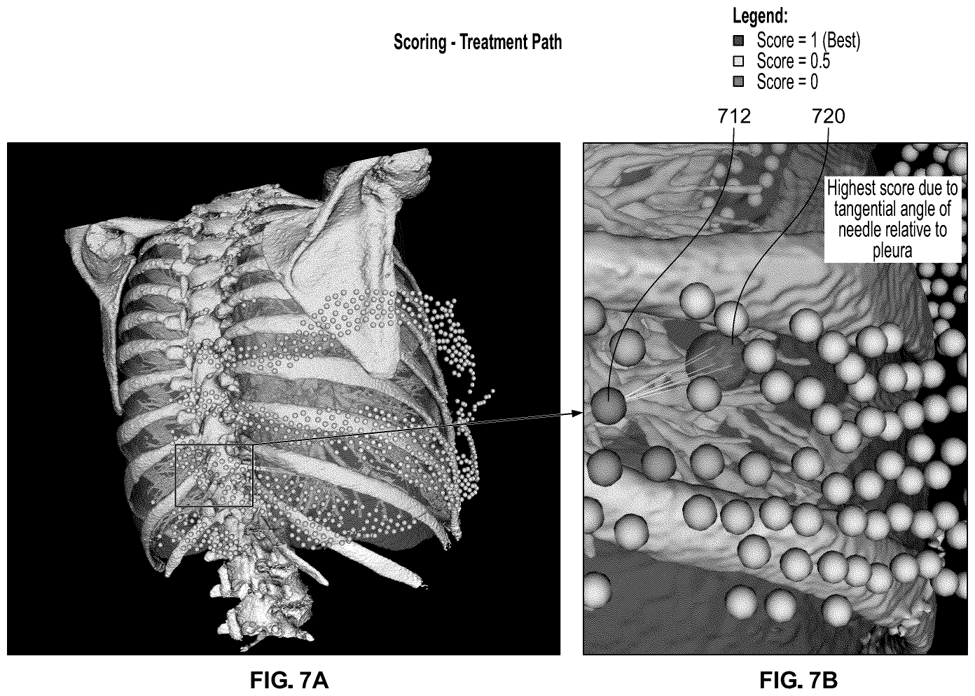

With reference to FIG. 7A, a treatment path score is visually shown for each skin location where best scores are shown according to the legend. The enlarged view shown in FIG. 7B illustrates a skin location 712 aimed at target 720. The skin location 712 has a high score due to the tangential angle of the electrode or device body relative to the pleura (not shown).

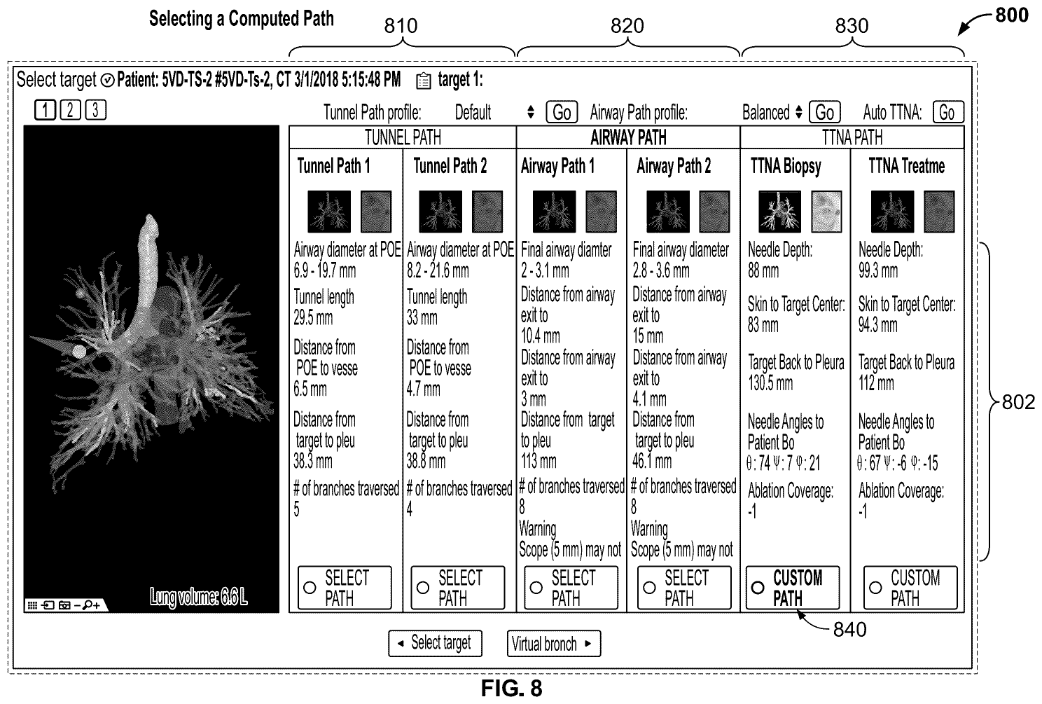

Optionally, and with reference to FIG. 8, a plurality of different types of paths 800 can be presented to the user. In the embodiment shown in FIG. 8, various components 802 computed for each path are also shown.

Tunnel paths 810 refer to a tunnel path to the target from a nearby airway having the highest scores. An example of tunnel path planning is described U.S. Pat. No. 8,709,034 to Keast et al., entitled “METHODS AND DEVICES FOR DIAGNOSING, MONITORING, OR TREATING MEDICAL CONDITIONS THROUGH AN OPENING THROUGH AN AIRWAY WALL” and as provided in the commercially available Archimedes® system manufactured by Broncus Medical, Inc. (San Jose, CA).

Airway paths 820 refer to a planned bronchoscopic route through the patient airways to the target having the highest scores. An example of airway path planning is described U.S. Pat. No. 9,675,420 to Higgins et al., entitled “METHODS AND APPARATUS FOR 3D ROUTE PLANNING THROUGH HOLLOW ORGANS” and as provided in Lungpoint® system manufactured by Broncus Medical, Inc. (San Jose, CA).

TTNA paths 830 refer to the biopsy path and treatment path having the highest scores. In this embodiment, for each of the biopsy and treatment path, the following computed route characteristics are displayed: needle depth, skin to target center, target back to pleura, needle angles to patient body (θ,ψ,φ), and ablation coverage (0 to 1).

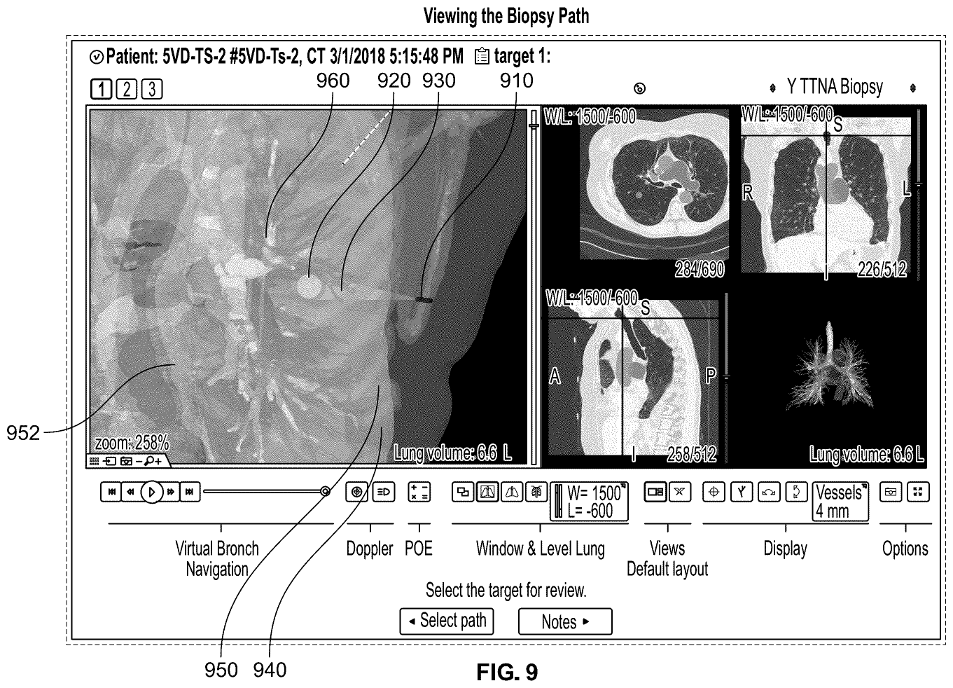

The system is programmed and operable to present tabs 840 for the user to select. The user can conveniently select a tab for displaying various virtual views of the selected route in a new window, discussed below in connection with FIGS. 9-12.

FIG. 9 is an enlarged fused CT view showing the 3D anatomical surfaces and biopsy path on the CT slice. The view shown in FIG. 9 includes a DRR with the skin location 910, target 920, route 930, pleura 940, ribs 950, spine 952, and vasculature 960 superimposed thereon. Optionally, several other views are provided including, for example, the traverse, coronal, sagittal CT or MRI views as well as a global virtual view of the airways and/or vasculature with the ribs removed for clarity.

Indeed, the system processor is programmed and operable to generate a wide variety of views for display, and to allow the user to zoom in, zoom out, change angles, drive through, add or remove layers or tissue types in real time. The views can be computed using a rendering engine based on the segmented anatomies and route computations, described above.

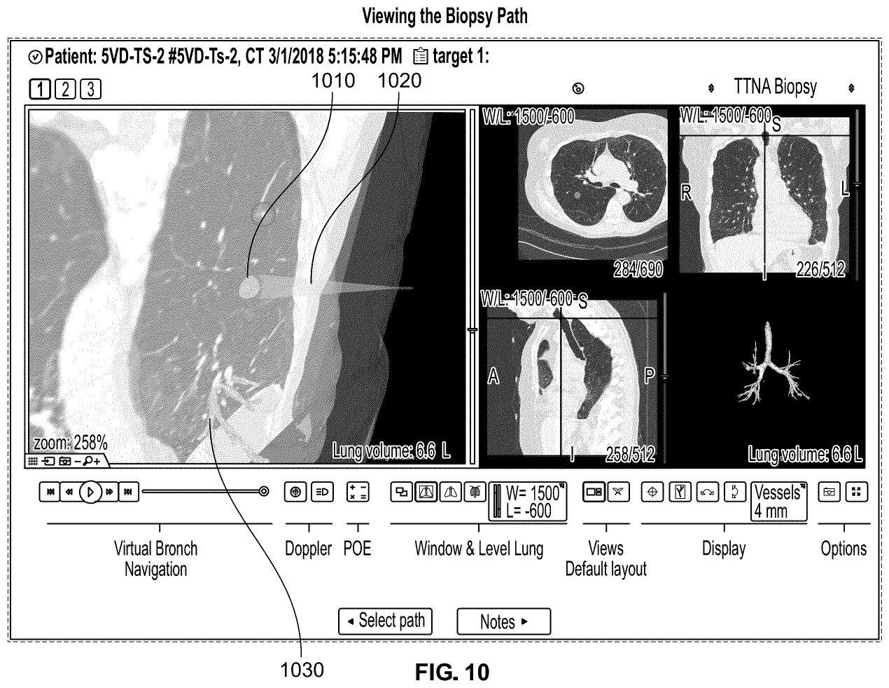

FIG. 10 is another fused CT view of the biopsy path shown in FIG. 9. In particular, FIG. 10 is a CT slice showing only the target 1010 and biopsy path 1020 and airway 1030 fused/superimposed thereon.

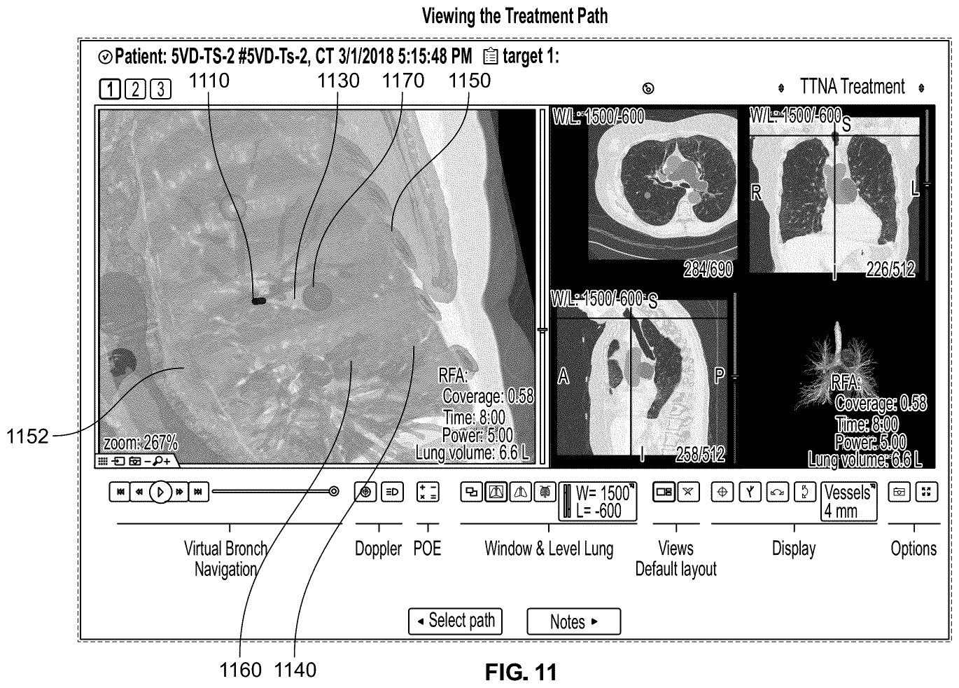

FIG. 11 is an enlarged CT view of the treatment path showing the 3D anatomy fused thereon. The view shown in FIG. 11 includes a CT slice with the skin location 1110, route 1130, pleura 1140, ribs 1150, spine 1152, vasculature 1160, and target/ablation zone 1170 superimposed thereon. Optionally, several other views are provided including, for example, the traverse, coronal, sagittal CT or MRI views as well as a global virtual view of the airways and/or vasculature with the ribs removed for clarity.

FIG. 12 is another fused CT view of a treatment path superimposed onto a CT view. In particular, FIG. 12 is a CT slice showing the treatment path 1210, airway 1220, and predicted ablation zone 1230 superimposed thereon. The ablation zone 1230 is shown having an ellipsoid shape, radiating from a longitudinal axis 1232 representing the major axis of an ablation electrode of ablation device. As described herein, in embodiments, the pleura 1240 is segmented and its distance from the target is computed. Where the distance is shortest (e.g., distance 1250 shown in FIG. 12), the angle between the electrode major axis 1232 and the pleura surface is evaluated. Skin locations that are operable to deploy the electrode at a minimum angle to the pleura are weighted higher. Stated alternatively, when the major axis of the electrode is parallel to the nearest pleura surface, the corresponding skin location is weighted higher. Optionally, several other views can be provided including, for example, the traverse, coronal, sagittal CT or MRI views as well as a global virtual view of the airways and/or vasculature with the ribs removed for clarity.

### Route Adjustment

Additionally, in embodiments, the skin location, minimum distance from obstacles, angle of attack, target boundary and center, route, and component weights may be modified by the physician. The computer is programmed and operable to recompute a new route based on the adjusted information. For example, an automatically-generated path may be adjusted by the physician. The auto-generated path can be used as a seed point for the physician to confirm the route, or make small adjustments. Providing a recommended seed point to the physician can save time and improve accuracy. All new skin locations, routes, and associated information (including a version number, ID, or time stamp) can be stored. One or more plans may be saved, displayed, and printed as desired by the physician.

Other modifications and variations can be made to the disclosed embodiments without departing from the subject invention.

We claim:

1. A method for automatically planning a transthoracic route from a candidate location on the skin of a patient to a target nodule in the patient on which to perform a procedure, said method comprising:
identifying one or more candidate skin locations on the skin of the patient based on a 3D model of a thoracic anatomy of the patient;
automatically computing a route to the target nodule from each candidate skin location; and
automatically computing a score for each candidate skin location for biopsy and for ablation, and wherein the computing is based on (a) route characteristics, (b) procedural parameters, and (c) obstacle clearance along the route.

2. The method of claim 1, wherein computing the route is based on minimizing the distance from the candidate skin location to the center of the target nodule.

3. The method of claim 1, wherein the route characteristics comprise a length of the route, and optionally, weighting the candidate skin locations higher based on minimizing the length of the route.

4. The method of claim 1, comprising computing the depth the needle is inserted from the skin for biopsy, and weighting the candidate skin locations higher based on minimizing the needle depth.

5. The method of claim 1, wherein the procedural parameters comprise orientation of an elongate ablation element relative to the pleura nearest the target nodule.

6. The method of claim 5, comprising weighting candidate skin locations for ablation higher based on minimizing an angle between the elongate ablation element and the pleura surface nearest the target nodule.

7. The method of claim 6, wherein the procedural parameters comprise a tissue ablation model, and optionally, a radio-frequency ablation empirical-based model.

8. The method of claim 7, wherein the tissue ablation model is ellipsoid-shaped.

9. The method of claim 1, comprising weighting candidate skin locations higher based on obstacle clearance along the route.

10. The method of claim 9, wherein the obstacles comprise bone, vessels, airways, lobe fissures, and heart.

11. The method of claim 1, wherein the target nodule is identified by the user.

12. The method of claim 1, further comprising displaying each candidate skin location for ablation and biopsy whose computed score is above a threshold level.

13. The method of claim 12, wherein the displaying includes visually indicating whether the candidate skin location is for ablation or biopsy.

14. The method of claim 1, wherein the score is further based on the patient's breathing motion.

15. The method of claim 1, wherein the target nodule is a lung tumor.

16. The method of claim 1, further comprising computing a final device position and orientation at center of mass of the target nodule relative to a coordinate system of the 3D model, and optionally displaying the final device position in a 3D view in combination with the target nodule.

17. The method of claim 1, further comprising computing an initial device position and orientation prior to commencing the thoracic procedure and relative to the coordinate system of the 3D model.

18. The method of claim 1, further comprising receiving an image data set of the thoracic anatomy, segmenting the thoracic anatomy to create a 3D model of a thoracic anatomy of the patient comprising the skin and the target nodule, and at least one of the following selected from the group comprising bone, blood vessels, pleura, airways, heart, lobes, fissures, and lymph nodes.

19. The method of claim 1, wherein the route is straight.

20. The method of claim 1, wherein the route is a cylindrical projection from the candidate skin location to the target nodule, and optionally having an outer diameter ranging from 1 to 2 mm.

21. The method of claim 1, further comprising determining the location of the target nodule center of mass, and wherein the computing the route is based on extending the route to the target nodule center of mass.

22. The method of claim 1, wherein spacing between adjacent candidate skin locations is about equal.

23. The method of claim 1, wherein the candidate skin location for ablation or biopsy can be adjusted by the physician.

24. The method of claim 1, wherein the route to a target nodule can be adjusted by the physician.

25. A system for automatically planning a transthoracic route from a candidate location on the skin of a patient to a target nodule in the patient on which to perform a procedure, said system comprising a processor programmed and operable to:
identify one or more candidate skin locations on the skin of the patient;
automatically compute a route to the target nodule from each candidate skin location; and
automatically compute a score for each candidate skin location for biopsy and for ablation, and wherein the computing is based on (a) route characteristics, (b) procedural parameters, and (c) obstacle clearance along the route.

26. The system of claim 25 further comprising a display operable with the processor to display at least one computed route and score for the computed route, and optionally, to display a fused arrangement of the route and segmented anatomy of the patient.

27. The system of claim 25, wherein the processor is further programmed and operable to segment a 3D model of a thoracic anatomy of the patient, the thoracic anatomy comprising the skin and the target nodule, and optionally, bone, blood vessels, pleura, airways, heart, lobes and lymph nodes.

28. The system of claim 25, wherein the processor is further programmed and operable to select a first-tier candidate skin location based on its score, and to compute a set of refined candidate skin locations, each of which is within a predetermined spacing from the first tier candidate location, and to automatically compute a score for each refined candidate skin location for biopsy and for ablation, and wherein the computing is based on (a) route characteristics, (b) procedural parameters, and (c) obstacle clearance along the route.

29. The system of claim 25, wherein the processor is further programmed and operable to score skin locations by weighting candidate skin locations for ablation higher based on minimizing an angle between a major axis of an ablation element and the pleura surface nearest the target nodule.

30. A system for automatically planning a transthoracic route from a candidate location on the skin of a patient to a target nodule in the patient on which to perform a procedure, said system comprising a processor programmed and operable to:
identify one or more candidate skin locations on the skin of the patient;
automatically compute a route to the target nodule from each candidate skin location; and
automatically provide feedback for each candidate skin location for biopsy and for ablation, and wherein the computing is based on (a) route characteristics, (b) procedural parameters, and (c) obstacle clearance along the route.
